(19) United States
(12) Patent Application Publication
(10) Pub. No.: US 2023/0186173 A1
(43) Pub. Date: Jun. 15, 2023

(54) METHOD OF ANALYZING INFLUENCE FACTOR FOR PREDICTING CARBON DIOXIDE CONCENTRATION OF ANY SPATIOTEMPORAL POSITION

(71) Applicant: Wuhan University, Wuhan (CN)

(72) Inventors: Zhaocong WU, Wuhan (CN); Lu MO, Wuhan (CN); Zhao YAN, Wuhan (CN)

(21) Appl. No.: 17/882,649

(22) Filed: Aug. 8, 2022

(30) Foreign Application Priority Data
Dec. 14, 2021 (CN) 202111524281.7

(51) Int. Cl. G06N 20/20; G01N 33/00

(52) U.S. Cl. CPC G06N 20/20; G01N 33/0039

(57) ABSTRACT

The disclosure provides a method of analyzing an influence factor for predicting a carbon dioxide concentration of any spatiotemporal position. Firstly, an atmospheric carbon dioxide spatiotemporal distribution simulation method is proposed. This simulation method constructs a simulation model simulating carbon dioxide concentration distribution of any position of a region based on machine learning algorithm in combination with carbon dioxide data of satellite observation and corresponding environmental factors; next, by use of a global sensitivity analysis method, quantitative evaluation on the importance of multiple influence factors for regional carbon dioxide distribution is achieved.

### CROSS-REFERENCE TO RELATED APPLICATIONS

Pursuant to 35 U.S.C. § 119 and the Paris Convention Treaty, this application claims foreign priority to Chinese Patent Application No. 202111524281.7 filed Dec. 14, 2021, the contents of which, including any intervening amendments thereto, are incorporated herein by reference. Inquiries from the public to applicants or assignees concerning this document or the related applications should be directed to: Matthias Scholl P.C., Attn.: Dr. Matthias Scholl Esq., 245 First Street, 18th Floor, Cambridge, Mass. 02142.

### BACKGROUND

The disclosure relates to the field of remote-sensing monitoring of greenhouse gas, and more particularly to a machine-learning-based atmospheric carbon dioxide spatiotemporal distribution simulation, and a global sensitivity analysis method of an influence factor.

Carbon dioxide is a major contributor to greenhouse gas and global warming and therefore, accurately knowing about the spatiotemporal distribution of carbon dioxide concentration and its changing trend is greatly important for knowing about and mitigating greenhouse effect. Satellite observation can accurately provide ground carbon dioxide information with a given spatiotemporal resolution and relatively long time sequence observation. Due to influence of clouds and aerosols and the like, there may be gaps in the satellite observation data in actual applications and thus it is difficult to accurately analyze the regional carbon dioxide spatiotemporal distribution. A common method of obtaining a carbon dioxide concentration of any position of a region is interpolation, including interpolation of spatial domain and interpolation for building a physical model based on carbon dioxide time sequence law, where the spatial domain interpolation method has a low accuracy and the physical model interpolation method generates a very complex model and has a low computing efficiency. Along with continuous development of machine learning algorithm, relevant researches on application of various neural network and machine learning models to a regional CO₂ simulation modeling so as to compensate a spatiotemporal gap of XCO2 data also starts developing. In this way, a high accuracy and wide-scope carbon dioxide spatiotemporal distribution graph can be efficiently generated. However, the existing machine learning-based methods usually only consider an aspect of the environmental factor or the anthropogenic emission factor for modeling, and the carbon dioxide concentration is subjected to influence of both of them. At present, a relevant method is still needed.

Furthermore, regional carbon dioxide distribution is affected by many factors such as natural environment and anthropogenic emission. The influence factors are complex, the process is complex and there are many relevant researches. But, most of them are qualitative analysis or relevance analysis of environmental factor or carbon dioxide concentration and quantitative evaluation methods for multi-factor influence degree are fewer. Thus, quantitative analysis for the contribution and influence of different environmental factors on atmospheric carbon dioxide concentration cannot be achieved.

### SUMMARY

The disclosure aims to provide a machine-learning-based atmospheric carbon dioxide spatiotemporal distribution simulation, and a global sensitivity analysis method of an influence factor. In this way, simulation can be achieved for a region without carbon dioxide data of satellite observation to obtain a carbon dioxide spatiotemporal distribution mode of the entire region and the importance degree of an environmental factor affecting regional carbon dioxide distribution is quantized by using the global sensitivity analysis for the model.

To achieve the above purpose, the technical solution adopted by the disclosure provides a method of analyzing an influence factor for predicting a carbon dioxide concentration of any spatiotemporal position. Firstly, an atmospheric carbon dioxide spatiotemporal distribution simulation method is proposed. This simulation method constructs a simulation model simulating carbon dioxide concentration distribution of any position of a region based on machine learning algorithm in combination with carbon dioxide data of satellite observation and corresponding environmental factors; next, by use of a global sensitivity analysis method, quantitative evaluation on the importance of multiple influence factors for regional carbon dioxide distribution is achieved. The method comprises the following steps:

at step 1, in combination with regional environmental characteristics, classifying environmental factors affecting regional carbon dioxide distribution into, comprising but not limited to, a ground and vegetation coverage factor, a climate meteorological factor, and a combustion emission factor;

at step 2, in combination with satellite carbon dioxide observation data and environmental factors, using machine learning algorithm to construct a regional carbon dioxide spatiotemporal distribution simulation model and training the simulation model using a training dataset;

at step 3, for the constructed carbon dioxide spatiotemporal distribution simulation model, firstly using a test dataset to verify a model prediction accuracy, and then inputting environmental factor data without satellite observation into the trained carbon dioxide spatiotemporal distribution simulation model to obtain a predicted carbon dioxide concentration and finally obtaining a regional carbon dioxide concentration distribution graph;

at step 4, in combination with the constructed regional carbon dioxide spatiotemporal distribution simulation model and the global sensitivity analysis method, calculating a sensitivity of the carbon dioxide concentration for each input parameter, i.e. environmental factor;

at step 5, counting the sensitivities of the regional carbon dioxide concentration for different environmental factors obtained by the global sensitivity analysis method, and quantitatively analyzing the size of the sensitivity of each parameter to finally determine an influence degree of each environmental factor along with the regional carbon dioxide distribution.

Furthermore, in step 1, the environmental factor classification specifically comprises ground coverage type, vegetation coverage, climate type, precipitation, atmospheric temperature, wind velocity and direction, anthropogenic emission amount, and biomass combustion emission amount of a region.

The vegetation coverage is represented by normalization vegetation index data which may be obtained from the L3 vegetation index product of the MODIS satellite; the anthropogenic emission statistics come from the high resolution global anthropogenic emission dataset ODIAC; the biomass combustion data comes from global fire disaster emission database GFED4; atmospheric temperature and precipitation data comes from Chinese 1 km-resolution monthly average atmospheric temperature dataset provided by National Tibetan Plateau Data Center; the ground coverage data comes from annual global land coverage dataset published by European Space Agency, the climate type data comes from Köppen climate zoning dataset, and the wind velocity and direction data comes from ERAS dataset.

Further, the machine learning algorithm used in step 2 is eXtreme Gradient Boosting tree (XGBoost) which is a tree integration model based on gradient boost, wherein the basic construction thinking of the model is: firstly constructing an initial sub-tree to performing fitting for data to correspondingly obtain a fitting residue, and constructing subsequent sub-trees based on previous model residue until the model residue is less than a threshold, and the final simulation result is a sum of all sub-tree results; the specific construction steps are as follows:

initially constructing a weak learner to obtain a residue corresponding to an initial model;

for each subsequent training iteration, based on the existing model, adding one weak learner to fit a residue of a previous model;

through continuous learning, fitting K weak learners to reduce the residue between a model prediction result and a true value until the residue is less than a threshold, and the model is terminated, where the final model prediction value is a result obtained by performing weighted summing using K base learners.

Further, the specific implementation of performing training using the training dataset in step 2 is as follows:

first performing preprocessing for the training dataset, comprising data cleaning, data encoding and data transformation, wherein the data cleaning comprises removal of missing value, abnormal value and noise, and the data transformation comprises normalization and dimension reduction;

wherein the data encoding is to encode non-numerical features and input into the model for training, that is, encode the environmental factors i.e. the ground vegetation type, the climate type and wind direction, by using one-hot encoding;

performing normalization processing for the data in the following formula:

$${z}_{q}^{′} = \frac{{z}_{q} - mean ( {z}_{q} )}{std ( {z}_{q} )}$$

wherein mean(z_{q}) is a mean value of data of environmental factor z_{q}, and std(z_{q}) is a standard deviation of the data of the environmental factor z_{q};

next, inputting the preprocessed training dataset into the XGBoost model and performing parameter adjustment and further optimization for the XGBoost model, and repeating iterations to obtain an optimal carbon dioxide spatiotemporal distribution simulation model.

Further, the base learner of the XGBoost model is CART tree, and for a dataset with m features of n samples D=(xᵢ,yᵢ) (|D|=n,xᵢ∈R^{m},yᵢ∈R), the final prediction value obtained by training is expressed below:

$${\hat{y}}_{i} = φ ( {x}_{i} ) = {\sum }_{k = 1}^{K} {f}_{k} ( {x}_{i} )$$

wherein K is a number of base learners, xᵢ is an i-th sample, yᵢ is a class label corresponding to the i-th sample, fₖ(⋅) is a model of a k-th tree, wherein the k-th tree is split into a leave node q of the tree and a corresponding weight part ω, i.e.:

fᵢ(xᵢ)=ω_{q(x}_{ᵢ}₎

wherein ω_{q(x}_{ᵢ}₎ is a weight of the leave node q where the sample xᵢ is located, and q(xᵢ) is a position of the leave node where the sample xᵢ is located, that is, for any one sample xᵢ, the weight at a particular leave node is valued as ω_{q(x}_{ᵢ}₎;

for each iteration, the model fits the previous predicted residue and therefore, when a t-th base learner is generated, the prediction model is expressed as:

ŷᵢ^{(t)}=ŷᵢ^{(t-1)}+fₖ^{(t)}(xᵢ)

a target function is expressed as:

$${L}^{( t )} = {\sum }_{i = 1}^{n} l ( {y}_{i} , ( {\hat{y}}_{i}^{( t - 1 )} + {f}_{k}^{( t )} ( {x}_{i} ) ) ) + Ω ( {f}_{k}^{( t )} )$$

wherein the target function is composed of two parts: in a first part, function l(⋅,⋅) describes a difference between a true value and a fitting value, which is calculated based on Euclidean distance; the second part is a regularized part Ω(fₖ^{(t)}) for preventing function overfitting, i.e.

$$Ω ( {f}_{k}^{( t )} ) = γ T + \frac{1}{2} {λΣ}_{j = 1}^{T} {ω}_{j}^{2}$$

used to limit the complexity of each tree and prevent model overfitting, wherein T is a number of all leave nodes on the CART tree, γ and λ are hyperparameters used to adjust the number of leave nodes and importance distribution of the weight during regularized calculation, ωⱼ is a weight value of a j-th leave node; to minimize the target function, the XGBoost considers performing second order Taylor expansion for the target function, which is approximately expressed as:

$${L}^{( t )} ≅ {\sum }_{i = 1}^{n} [ l ( {y}_{i} , {\hat{y}}_{i}^{( t - 1 )} ) + {g}_{i} {f}_{k}^{( t )} ( {x}_{i} ) + \frac{1}{2} {h}_{i} {f}_{k}^{{( t )}^{2}} ( {x}_{i} ) ] + γ T + \frac{1}{2} λ {\sum }_{j = 1}^{T} {ω}_{j}^{2}$$

wherein gᵢ is a first-order derivative, defined as

$${ℊ}_{i} = {\partial }_{{\hat{y}}_{i}^{( t - 1 )}} l ( {y}_{i} , {\hat{y}}_{i}^{( t - 1 )} )$$

hᵢ is a second-order derivative

$${h}_{i} = {\partial }_{{\hat{y}}_{i}^{( t - 1 )}}^{2} l ( {y}_{i} , {\hat{y}}_{i}^{tc - 1 )} ) ,$$

and the following result is obtained by substituting into the target function:

$${L}^{( T )} ≅ {\sum }_{j = 1}^{T} [ ( {\sum }_{i = 1} {g}_{i} ) {ω}_{j} + \frac{1}{2} ( {\sum }_{i = 1} {h}_{i} + λ ) {ω}_{j}^{2} ]$$

each iteration minimizes the target function to obtain j optimal leave nodes of the t-th base learner and an optimal solution ωₗ corresponding to each leave node.

Further, the global sensitivity analysis method used in step 4 is Sobol method, the sensitivity of which is calculated by decomposing an output total variance into a sum of a variance of each parameter and a variance of mutual interaction of parameters, and then performing sensitivity grading calculation based on a ratio of a contribution of the parameter to the output variance;

for each environmental factor, a change range and a probability distribution are calculated and then a corresponding sensitivity index is calculated in combination with the regional carbon dioxide spatiotemporal distribution simulation model;

the regional carbon dioxide spatiotemporal distribution simulation model is expressed as: y=f(x₁′,x₂′, . . . , xₚ′), wherein f is a trained XGBoost model, x₁′,x₂′, . . . , xₚ′ are environmental factors affecting carbon dioxide distribution and are input parameters of the XGBoost model; the total variance of the XGBoost model is:

D=∫f²(x′)dx′−f₀²

wherein, f₀ is an initial value of the model and the a partial variance of the XGBoost model is:

D_{π}_{₁}_{,π}_{₂}_{, . . . ,π}_{ₛ}=∫ . . . ∫(x_{π}_{₁}′,x_{π}_{₂}′, . . . ,x_{π}_{ₛ}′)dx_{π}_{₁}′,x_{π}_{₂}′, . . . ,x_{π}_{ₛ}′

wherein, 1≤π₁< . . . <πₛ≤p, and s=1, 2, . . . , p and the sensitivity S_{π}_{₁}_{,π}_{₂}_{, . . . ,π}_{ₛ} of each environmental factor:

$${S}_{{π}_{1} , {π}_{2} , … , {π}_{s}} = \frac{{D}_{{π}_{1} , {π}_{2} , … , {π}_{s}}}{D}$$

wherein S_{π}_{₁} is a first-order sensitivity index of the environmental factor x_{π}_{₁}′, which is used to represent an influence of the parameter on the model output, S_{π}_{₁}_{,π}_{₂}_{, . . . , π}_{ₛ} is an s-order sensitivity index of the environmental factors x_{π}_{₁}′,x_{π}_{₂}′, . . . , x_{π}_{ₛ}′, which is used to represent a joint influence of s parameters on the model;

further, a total sensitivity index of each environmental factor is obtained, and the total sensitivity index TS_{π} of the environmental factor x_{π}_{ₛ}′ is defined as:

TS_{π}=S_{π}_{₁}+S_{π}_{₁}_{,π}_{₂}+ . . . +S_{π}_{₁}_{,π}_{₂}_{, . . . ,π}_{ₛ}

the total sensitivity index of each environmental factor obtained by Sobol method is used to evaluate the final sensitivity of the influence factors affecting the regional carbon dioxide distribution, achieving quantitative influence degree analysis.

Compared with the prior art, the disclosure has the following advantages and beneficial effects.

In the disclosure, when retrieval is performed for a regional carbon dioxide distribution, a machine learning model is built by making full consideration for all ground environments, climatic meteorology and anthropogenic combustion emission factors relating to carbon dioxide concentration, thus achieving more accurate and quicker prediction for the regional carbon dioxide concentration distribution. Further, based on the built machine learning model, in a case of consideration of interactive effect, the sensitivity of each factor affecting the regional CO2 growth is quantitatively evaluated so as to provide scientific guidance for formulation of carbon emission policy.

### BRIEF DESCRIPTION OF THE DRAWINGS

FIG. 1 is a flowchart illustrating a general method of an embodiment of the disclosure;

FIG. 2A is a regional carbon dioxide distribution graph of satellite carbon dioxide observation data and FIG. 2B is modeling retrieval according to an embodiment of the disclosure; and

FIGS. 3A-3B are sector graph of a sensitivity index of an influence factor of an embodiment of the disclosure.

### DETAILED DESCRIPTION

To describe the technical solution and technical advantages of the disclosure in more details, the disclosure will be fully described below in combination with specific embodiments and accompanying drawings.

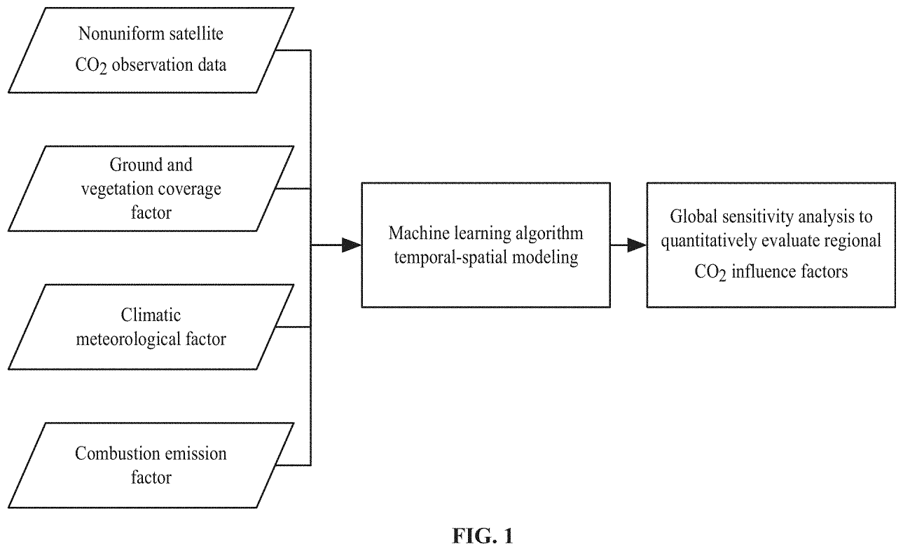

As shown in FIG. 1, the disclosure provides a method of analyzing an influence factor for predicting a carbon dioxide concentration of any spatiotemporal position. The method generally comprises two parts. In a first part, regional carbon dioxide simulation modeling is performed based on machine learning algorithm to achieve simulation for a region without satellite observation carbon dioxide data so as to obtain a carbon dioxide spatiotemporal distribution mode of the entire region; in a second part, according to a trained regional carbon dioxide spatiotemporal distribution simulation model, in combination with a global sensitivity analysis method, an importance degree of an environmental factor affecting regional carbon dioxide distribution is quantized. The specific implementation process is described below.

1. The specific steps of the regional carbon dioxide simulation modeling method based on machine learning algorithm are described below.

At step 1, environmental factor data affecting regional carbon dioxide distribution are collected, including but not limited to ground coverage type, vegetation coverage, climate type, precipitation, atmospheric temperature, wind velocity and direction, anthropogenic emission amount statistic data, and biomass combustion emission amount of a region, and then matched with the satellite observation carbon dioxide data to obtain training and verification datasets of a machine learning model.

The vegetation coverage is represented by normalization vegetation index data which may be obtained from the L3 vegetation index product of the MODIS satellite; the anthropogenic emission statistics come from the high resolution global anthropogenic emission dataset ODIAC; the biomass combustion data comes from global fire disaster emission database GFED4; atmospheric temperature and precipitation data comes from Chinese 1 km-resolution monthly average atmospheric temperature dataset provided by National Tibetan Plateau Data Center; the ground coverage data comes from annual global land coverage dataset published by European Space Agency, the climate type data comes from Köppen climate zoning dataset, and the wind velocity and direction data comes from ERAS dataset.

At step 2, a machine learning algorithm is selected to construct a carbon dioxide distribution simulation model and the model is trained in combination with environmental factors and the satellite carbon dioxide training dataset.

The specific steps of performing training are as follows: preprocessing the training dataset, comprising data cleaning (removal of missing value, abnormal value and noise and the like), data encoding and data transformation (normalization and dimension reduction and the like) and so on.

For the processing of the missing value of the dataset, in a case of less missing values, it is considered to delete the sample.

For the processing for abnormal value and noise, the noise is firstly detected by statistic characteristics of data or clustering method, and then the data is “smoothed” by using a method such as binning, clustering, regression, and combination of computer check and manual check to remove the abnormal values and noise in the data.

The data encoding is mainly to encode the non-numerical features and input them into the model for training. In this experiment, it is mainly required to encode the environmental factors such as ground coverage type, climate type and wind direction, by using one-hot encoding.

Data preprocessing also requires normalization processing for the data in the following formula:

$${z}_{q}^{′} = \frac{{z}_{q} - mean ( {z}_{q} )}{s t d ( {z}_{q} )}$$

where mean(z_{q}) is a mean value of data of environmental factor z_{q}, and std(z_{q}) is a standard deviation of the data of the environmental factor z_{q}.

Furthermore, the machine learning algorithm used in step 2 is eXtreme Gradient Boosting tree (XGBoost) which is a tree integration model based on gradient boost, wherein the basic construction thinking of the model is: firstly constructing an initial sub-tree to performing fitting for data to correspondingly obtain a fitting residue, and constructing subsequent sub-trees based on previous model residue until the model residue is less than a threshold, and the final simulation result is a sum of all sub-tree results; the specific construction steps are as follows:

initially constructing a weak learner to obtain a residue corresponding to an initial model;

for each subsequent training iteration, based on the existing model, adding one weak learner to fit a residue of a previous model;

through continuous learning, fitting K weak learners to reduce the residue between a model prediction result and a true value until the residue is less than a threshold, and the model is terminated where the final model prediction value is a result obtained by performing weighted summing using K base learners.

Furthermore, the base learner of the XGBoost model is CART tree, and for a dataset with m features of n samples D=(xᵢ,yᵢ)(|D|=n,xᵢ∈R^{m},yᵢ∈R), the final prediction value obtained by training is expressed below:

$${\hat{y}}_{i} = φ ( {x}_{i} ) = {\sum }_{k = 1}^{K} {f}_{k} ( {x}_{i} )$$

wherein K is a number of base learners, xᵢ is an i-th sample, yᵢ is a class label corresponding to the i-th sample, fₖ(⋅) is a model of a k-th tree, wherein the k-th tree is split into a leave node q of the tree and a corresponding weight part co, i.e.:

fᵢ(xᵢ)=ω_{q(x}_{ᵢ}₎

wherein ω_{g(x}_{ᵢ}₎ is a weight of the leave node q where the sample xᵢ is located, and q(xᵢ) is a position of the leave node where the sample xᵢ is located, that is, for any one sample xᵢ, the weight at a particular leave node is valued as ω_{q(x}_{ᵢ}₎;

for each iteration, the model fits the previous predicted residue and therefore, when a t-th base learner is generated, the prediction model is expressed as:

ŷᵢ^{(t)}=ŷᵢ^{(t-1)}+fₖ^{(t)}(xᵢ)

a target function is expressed as:

$${L}^{( t )} = {\sum }_{i = 1}^{n} l ( {y}_{i} , ( {\hat{y}}_{i}^{( t - 1 )} + {f}_{k}^{( t )} ( {x}_{i} ) ) ) + Ω ( {f}_{i}^{( t )} )$$

wherein the target function is composed of two parts: in a first part, function l(⋅,⋅) describes a difference between a true value and a fitting value, which is calculated based on Euclidean distance; the second part is a regularized part Ω(fₖ^{(t)}) for preventing function overfitting, i.e.

$$Ω ( {f}_{k}^{( t )} ) = γ T + \frac{1}{2} λ {\sum }_{j = 1}^{T} {ω}_{j}^{2}$$

used to limit the complexity of each tree and prevent model overfitting, wherein T is a number of all leave nodes on the CART tree, γ and λ are hyperparameters used to adjust the number of leave nodes and importance distribution of the weight during regularized calculation, ωⱼ is a weight value of a j-th leave node; to minimize the target function, the XGBoost considers performing second order Taylor expansion for the target function, which is approximately expressed as:

$${L}^{( t )} ≅ {\sum }_{i = 1}^{n} [ l ( {y}_{i} , {\hat{y}}_{i}^{( t - 1 )} ) + {g}_{i} {f}_{k}^{( t )} ( {x}_{i} ) + \frac{1}{2} {h}_{i} {f}_{i}^{{( t )}^{2}} ( {x}_{i} ) ] + γ T + \frac{1}{2} λ {\sum }_{j = 1}^{T} {ω}_{j}^{2}$$

wherein gᵢ is a first-order derivative, defined as

$${ℊ}_{i} = {\partial }_{{\hat{y}}_{i}^{( t - 1 )}} l ( {y}_{i} , {\hat{y}}_{i}^{( t - 1 )} )$$

hᵢ is a second-order derivative

$${h}_{i} = {\partial }_{{\hat{y}}_{i}^{( t - 1 )}}^{2} l ( {y}_{i} , {\hat{y}}_{i}^{( t - 1 )} ) ,$$

and the following result is obtained by substituting into the target function:

$${L}^{( t )} ≅ {\sum }_{j = 1}^{T} [ ( {\sum }_{i = 1} {ℊ}_{i} ) {ω}_{j} + \frac{1}{2} ( {\sum }_{i = 1} {h}_{i} + λ ) {ω}_{j}^{2} ] + λ T$$

Each iteration minimizes the target function to obtain j optimal leave nodes of the t-th base learner and an optimal solution ωⱼ corresponding to each leave node.

The preprocessed training dataset is input into the XGBoost model and parameter adjustment and further optimization are performed for the XGBoost model, and iterations are repeated to obtain an optimal carbon dioxide distribution simulation model.

At step 3, for the constructed carbon dioxide distribution simulation model, a test dataset is firstly used to verify a model prediction accuracy, and then environmental factor data without satellite observation is input into the trained carbon dioxide distribution simulation model to obtain a predicted carbon dioxide concentration and finally, a regional carbon dioxide concentration spatiotemporal distribution is obtained.

2. According to the above trained regional carbon dioxide spatiotemporal distribution simulation model and the global sensitivity analysis method, the importance of the influence factors is quantitatively analyzed, comprising the following steps.

At step 4, in combination with the constructed regional carbon dioxide spatiotemporal distribution simulation model and the global sensitivity analysis method, a sensitivity of the carbon dioxide distribution for each environmental factor is calculated.

At step 5, the sensitivities of the regional carbon dioxide concentration for different environmental factors obtained by the global sensitivity analysis method are counted, and the size of the sensitivity of each parameter is quantitatively analyzed to finally determine an influence degree of each environmental factor along with the regional carbon dioxide distribution.

The global sensitivity analysis method used in step 4 is Sobol method which is performed in the following step:

for each environmental factor, a change range and a probability distribution are calculated and then a corresponding sensitivity index is calculated in combination with the regional carbon dioxide spatiotemporal distribution simulation model.

The regional carbon dioxide spatiotemporal distribution simulation model is expressed as: y=f(x₁′,x₂′, . . . , xₚ′), wherein f is a trained XGBoost model, x₁′,x₂′, . . . , xₚ′ are environmental factors affecting carbon dioxide distribution and are input parameters of the XGBoost model and n is a number of model parameters, i.e. the 9 influence factors in step 1; the total variance of the XGBoost model is:

D=∫f²(x′)dx′−f₀²

wherein, f₀ is an initial value of the model and the a partial variance of the model is:

D_{π}_{₁}_{,π}_{₂}_{, . . . ,π}_{ₛ}=∫ . . . ∫(x_{π}_{₁}′,x_{π}_{₂}′, . . . ,x_{π}_{ₛ}′)dx_{π}_{₁}′,x_{π}_{₂}′, . . . ,x_{π}_{ₛ}′

wherein, 1≤π₁< . . . <πₛ≤p, and s=1, 2, . . . , p and the sensitivity S_{π}_{₁}_{,π}_{₂}_{, . . . , π}_{ₛ} of each environmental factor:

$${S}_{{π}_{1} , {π}_{2} , … , {π}_{s}} = \frac{{D}_{{π}_{1} , {π}_{2} , … , {π}_{s}}}{D}$$

wherein S_{π}_{₁} is a first-order sensitivity index of the environmental factor x_{π}_{₁}′, which is used to represent an influence of the parameter on the model output, S_{π}_{₁}_{,π}_{₂}_{, . . . , π}_{ₛ} is an s-order sensitivity index of the environmental factors x_{π}_{₁}′,x_{π}_{₂}′, . . . ,x_{π}_{ₛ}′, which is used to represent a joint influence of s parameters on the model;

further, a total sensitivity index of each environmental factor is obtained, and the total sensitivity index TS_{π} of the environmental factor x_{π}_{ₛ}′ is defined as:

TS_{π}=S_{π}_{₁}+S_{π}_{₁}_{,π}_{₂}+ . . . +S_{π}_{₁}_{,π}_{₂}_{, . . . ,π}_{ₛ}

In step 5, the total sensitivity index of each environmental factor obtained by Sobol method is used to evaluate the final sensitivity of the influence factors affecting the regional carbon dioxide distribution, achieving quantitative influence degree analysis.

### 3. Embodiment

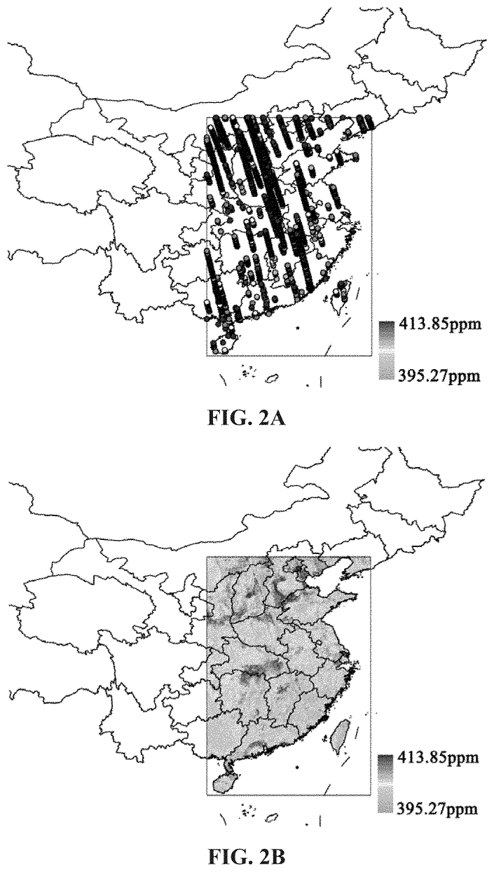

In this embodiment of the present disclosure, by using OCO-2 satellite XCO2 observation data and corresponding environmental factors of 2016 and the XGBoost modeling, the CO₂ concentration distribution in the eastern region of China is simulated. FIGS. 2A-2B show a result of satellite observation data and modeling retrieval. For accuracy evaluation on the simulation model constructed using machine learning algorithm, a determination coefficient R2 and a root mean square error RMSE are used and a final modeling accuracy obtained after parameter adjustment and optimization is as shown in Table 1.

TABLE 1
Modeling accuracy

| Training samples | Test samples | R2 | RMSE |
|---|---|---|---|
| 3153 (70%) | 1351 (30%) | 0.6751 | 1.6362 ppm |

By using the global sensitivity analysis method and the constructed carbon dioxide simulation model, quantitative evaluation is performed for the sensitivities of the influence factors to obtain the results as shown in Table 2.

TABLE 2
a first order sensitivity index and a total sensitivity index
of each environmental factor estimated
using the global sensitivity analysis method

| | First order | Total sensitivity |
|---|---|---|
| Environmental factors | sensitivity index | index |
| Ground coverage type | 0.013060 | 0.015529 |
| Vegetation coverage | 0.300257 | 0.320699 |
| Climate type | 0.006008 | 0.007367 |
| Precipitation | 0.291814 | 0.301615 |
| Atmospheric temperature | 0.262991 | 0.277399 |
| Wind velocity and direction | 0.713833 | 0.727576 |
| Anthropogenic emission | 0.000197 | 0.000208 |
| amount | | |
| Biomass combustion emission | 0.000915 | 0.001157 |

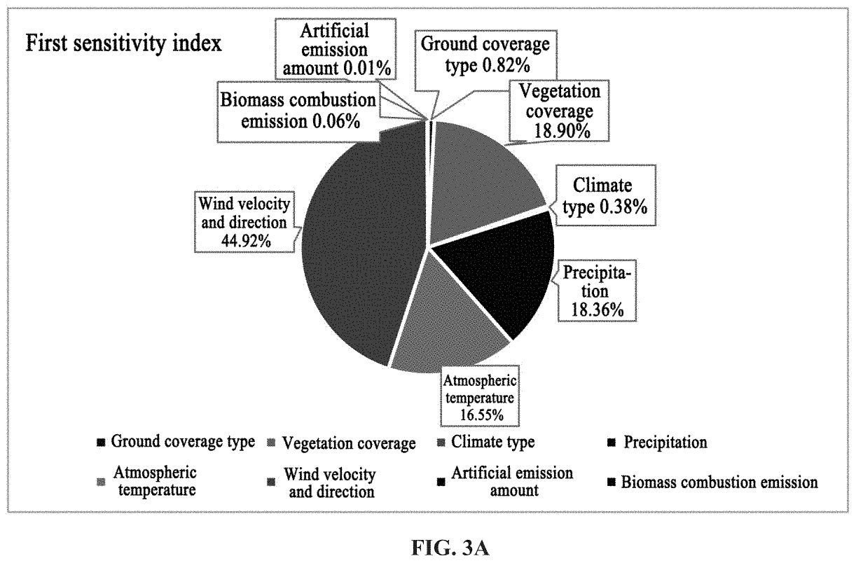

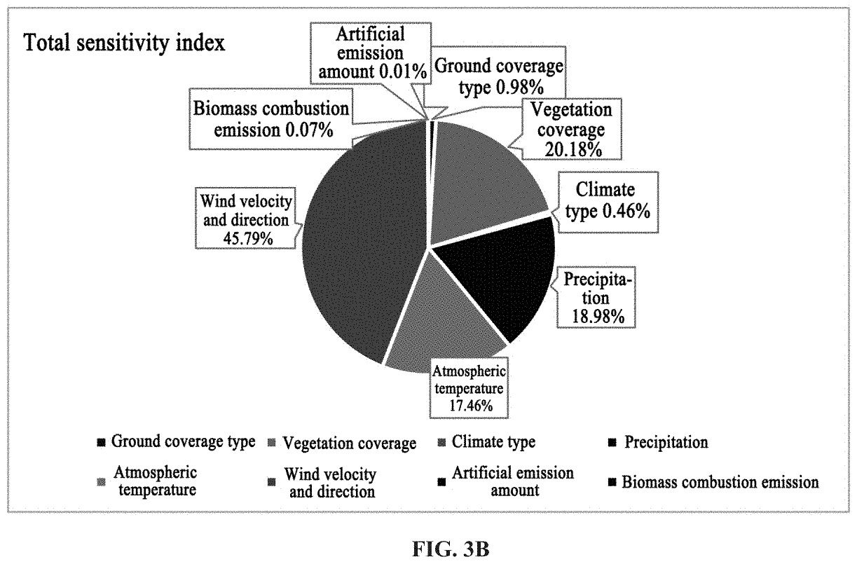

To more visually display the sizes of the sensitivities of different environmental factors on the total carbon dioxide distribution, a sector graph of sensitivity indexes is drawn to determine ratios of the influence factors as shown in FIGS. 3A-3B.

As shown in FIGS. 2A-2B, the environmental factors, i.e. wind velocity and direction, vegetation, precipitation, atmospheric temperature, ground coverage type, climate type, biomass combustion emission and anthropogenic emission, are sorted in a descending order of sensitivity size, where the indexes of the wind velocity and direction, vegetation, precipitation and atmospheric temperature are large, which indicates that they are major factors affecting regional carbon dioxide distribution.

As known from the model accuracy, it is feasible to simulate the regional carbon dioxide spatiotemporal distribution by using model. The method provided by the disclosure can fill in the gap of satellite observation data by simulating the regional carbon dioxide concentration spatiotemporal distribution with the environmental factors. Further, a method of quantitatively evaluating the influence degrees of the environmental factors on the regional carbon dioxide distribution is proposed so as to determine the influence sizes and specific degrees of various environmental factors on the regional carbon dioxide distribution.

The specific embodiments described in the disclosure are merely illustrated based on the spirit of the disclosure. Those skilled in the art can make various changes or supplementations or similar replacements to the specific embodiments described herein without departing from the spirit of the disclosure or the scope defined by the appended claims.

What is claimed is:

1. A method of analyzing an influence factor for predicting a carbon dioxide concentration of any spatiotemporal position, the method comprising:
1) in combination with regional environmental characteristics, classifying environmental factors affecting regional carbon dioxide distribution into a plurality of factors comprising ground coverage type factor, vegetation coverage factor, climate type factor, precipitation factor, atmospheric temperature factor, wind velocity and direction factors, anthropogenic emission amount factor, and biomass combustion emission factor;
wherein, in 1), the vegetation coverage factor is from the L3 Normalized Difference Vegetation Index of the Moderate-Resolution Imaging Spectroradiometer (MODIS) satellite; the ground coverage type factor is from the annual global land coverage data from European Space Agency; the climate type factor is from Köppen climate zoning dataset; the precipitation factor and the atmospheric temperature factor are from the Chinese 1 km-resolution monthly average precipitation and atmospheric temperature data from National Tibetan Plateau Data Center; the wind velocity and direction factors are from the wind velocity and direction data from ERAS dataset; the biomass combustion emission factor is from the anthropogenic emission amount from the high resolution global anthropogenic emission dataset ODIAC and biomass combustion emission amount data from the global fire disaster emission database GFED4;
2) in combination with OCO-2 satellite carbon dioxide observation data and the environmental factors, using eXtreme Gradient Boosting tree (XGBoost) machine learning algorithm to construct a Regional Carbon Dioxide Spatiotemporal distribution simulation (RCDS) model and training the simulation model using a training dataset;
3) for the constructed RCDS model, first using a test dataset to verify a model prediction accuracy, and then inputting environmental factor data without satellite observation into the trained carbon dioxide spatiotemporal distribution simulation model to obtain a predicted carbon dioxide concentration and finally obtaining a regional carbon dioxide concentration distribution graph;
4) in combination with the constructed regional carbon dioxide spatiotemporal distribution simulation model and a global sensitivity analysis method, calculating a sensitivity of the carbon dioxide concentration for each input environmental factor parameter;
5) counting the sensitivities of the regional carbon dioxide concentration for different environmental factors obtained by the global sensitivity analysis method, and quantitatively analyzing the size of the sensitivity of each parameter to finally determine an influence degree of each environmental factor along with the regional carbon dioxide distribution.

2. The method of claim 1, wherein the machine learning algorithm used in 2) is eXtreme Gradient Boosting tree (XGBoost) which is a tree integration model based on gradient boost; the basic construction thinking of the XGBoost model is: firstly constructing an initial sub-tree to performing fitting for data to correspondingly obtain a fitting residue, and constructing subsequent sub-trees based on initial sub-tree fitting residue until the subsequent sub-tree fitting residue is less than a threshold, and the final simulation result is a sum of all sub-tree results; the specific construction steps are as follows:
initially constructing a weak base learner to obtain a residue corresponding to an initial sub-tree model;
for each subsequent training iteration, based on the existing sub-tree model, adding one weak learner to fit a residue of a previous sub-tree model;
through continuous learning, fitting K weak learners to reduce the residue between a model prediction result and a true value until the residue is less than a threshold, and the model is terminated, finally the model prediction result is a result obtained by performing weighted summing using K base learners.

3. The method of claim 1, wherein, the specific implementation of performing training using the training dataset in 2) is as follows:
first performing preprocessing for the training dataset, comprising data cleaning, data encoding and data transformation, wherein the data cleaning comprises removal of missing value, abnormal value and noise, and the data transformation comprises normalization and dimension reduction;
the data encoding is to encode non-numerical features and input into the model for training, encode the environmental factors comprising the ground vegetation type, the climate type and wind direction, by using one-hot encoding;
performing normalization processing for the data in the following formula:
$${z}_{q}^{′} = \frac{{z}_{q} - mean ( {z}_{q} )}{std ( {z}_{q} )}$$
wherein mean(z_{q}) is a mean value of data of environmental factor z_{q}, and std(z_{q}) is a standard deviation of the data of the environmental factor z_{q};
next, inputting the preprocessed training dataset into the XGBoost model and performing parameter adjustment and further optimization for the XGBoost model, and repeating iterations to obtain an optimal carbon dioxide spatiotemporal distribution simulation model.

4. The method of claim 2, wherein the base learner of the XGBoost model is CART tree, and for a dataset with m features of n samples D=(xᵢ,yᵢ)(|D|=n, xᵢ∈R^{m}, yᵢ∈R), the final CART tree prediction value obtained by training is expressed below:
$${\hat{y}}_{i} = φ ( {x}_{i} ) = {\sum }_{k = 1}^{K} {f}_{k} ( {x}_{i} )$$
wherein K is a number of base learners, xᵢ is an i-th sample, yᵢ is a class label corresponding to the i-th sample, fₓ(⋅) is a model of a k-th tree; the k-th tree is split into a leave node q of the CART tree and a corresponding weight part ω, i.e.:
fᵢ(xᵢ)=ω_{q(x}_{ᵢ}₎
wherein ω_{q(x}_{ᵢ}₎ is a weight of the leave node q where the sample xᵢ is located, and q(xᵢ) is a position of the leave node where the sample xᵢ is located, that is, for any one sample xᵢ, the weight at a particular leave node is valued as ω_{q(x}_{ᵢ}₎;
for each iteration, the model fits the previous predicted residue and therefore, when a t-th base learner is generated, the prediction model is expressed as:
ŷᵢ^{(t)}=ŷᵢ^{(t-1)}+fₖ^{(t)}(xᵢ)
a target function is expressed as:
$${L}^{( t )} = {\sum }_{i = 1}^{n} l ( {y}_{i} , ( {\hat{y}}_{i}^{( t - 1 )} + {f}_{k}^{( t )} ( {x}_{i} ) ) ) + Ω ( {f}_{k}^{( t )} )$$
wherein the target function contains two parts: a first part is function l(yᵢ,(ŷᵢ^{(t-1)}+fₖ^{(t)}(xᵢ))), which describes a difference between a true value and a fitting (t) value based on Euclidean distance; a second part is Ω(fₖ^{(t)}), which is a regularized part for preventing function overfitting, i.e.
$$Ω ( {f}_{k}^{( t )} ) = γ T + \frac{1}{2} λ {\sum }_{j = 1}^{T} {ω}_{j}^{2}$$
used to limit the complexity of each tree and prevent model overfitting; T is a number of all leave nodes on the CART tree, γ and λ are hyperparameters used to adjust the number of leave nodes and importance distribution of the weight during regularized calculation, ωⱼ is a weight value of a j-th leave node; to minimize the target function, the XGBoost considers performing second order Taylor expansion for the target function, which is approximately expressed as:
$${L}^{( t )} ≅ {\sum }_{i = 1}^{n} [ l ( {y}_{i} , {\hat{y}}_{i}^{( t - 1 )} ) + {ℊ}_{i} {f}_{k}^{( t )} ( {x}_{i} ) + \frac{1}{2} {h}_{i} {f}_{k}^{{( t )}^{2}} ( {x}_{i} ) ] + γ T + \frac{1}{2} λ {\sum }_{j = 1}^{T} {ω}_{j}^{2}$$
wherein gᵢ is a first-order derivative, defined as
$${ℊ}_{i} = {\partial }_{{\hat{y}}_{i}^{( t - 1 )}} l ( {y}_{i} , {\hat{y}}_{i}^{( t - 1 )} )$$
hᵢ is a second-order derivative
$${h}_{i} = {\partial }_{{\hat{y}}_{i}^{( t - 1 )}}^{2} l ( {y}_{i} , {\hat{y}}_{i}^{( t - 1 )} ) ,$$
and the following result is obtained by substituting into the target function:
$${L}^{( T )} ≅ {\sum }_{j = 1}^{T} [ ( {\sum }_{i = 1} {ℊ}_{i} ) {ω}_{j} + \frac{1}{2} ( {\sum }_{i = 1} {h}_{i} + λ ) {ω}_{j}^{2} ]$$
each iteration minimizes the target function to obtain j optimal leave nodes of the t-th base learner and an optimal solution ωⱼ corresponding to each leave node.

5. The method of claim 1, wherein the global sensitivity analysis method used in 4) is Sobol method, the sensitivity of which is calculated by decomposing an output total variance into a sum of a variance of each parameter and a variance of mutual interaction of parameters, and then performing sensitivity grading calculation based on a ratio of a contribution of the parameter to the output variance;
for each environmental factor, a change range and a probability distribution are calculated and then a corresponding sensitivity index is calculated in combination with the regional carbon dioxide spatiotemporal distribution simulation model;
the regional carbon dioxide spatiotemporal distribution simulation model is expressed as: y=f(x₁′, x₂′, . . . , xₚ′), wherein f is a trained XGBoost model, x₁′, x₂′, . . . , xₚ′ are environmental factors affecting carbon dioxide distribution and are input parameters of the XGBoost model; the total variance of the XGBoost model is:
D=∫f²(x′)dx′−f₀²
wherein, f₀ is an initial value of the XGBoost model and a partial variance of the XGBoost model is:
D_{π}_{₁}_{,π}_{₂}_{, . . . ,π}_{ₛ}=∫ . . . ∫(x_{π}_{₁}′,x_{π}_{₂}′, . . . ,x_{π}_{ₛ}′)dx_{π}_{₁}′,x_{π}_{₂}′, . . . ,x_{π}_{ₛ}′
wherein, 1≤π₁< . . . <πₛ≤p, and s=1, 2, . . . , p and the sensitivity S_{π}_{₁}_{,π}_{₂}_{, . . . ,π}_{ₛ} of each environmental factor:
$${S}_{{π}_{1} , {π}_{2} , … , {π}_{s}} = \frac{{D}_{{π}_{1} , {π}_{2} , … , {π}_{s}}}{D}$$
wherein S_{π}_{₁} is a first-order sensitivity index of the environmental factor x_{π}_{₁}′, which is used to represent an influence of the environmental factor x_{π}_{₁}′ on the model output, S_{π}_{₁}_{,π}_{₂}_{, . . . ,π}_{ₛ} is an s-order sensitivity index of the environmental factors x_{π}_{₁}′, x_{π}_{₂}′, . . . , x_{π}_{ₛ}′, which is used to represent a joint influence of s environmental factors on the model;
further, a total sensitivity index of each environmental factor is obtained, and the total sensitivity index TS_{π} of the environmental factor x_{π}_{ₛ}′ is defined as:
TS_{π}=S_{π}_{₁}+S_{π}_{₁}_{,π}_{₂}+ . . . +S_{π}_{₁}_{,π}_{₂}_{, . . . ,π}_{ₛ}
the total sensitivity index of each environmental factor obtained by Sobol method is used to evaluate the final sensitivity of the influence factors affecting the regional carbon dioxide distribution, achieving quantitative influence degree analysis.
